(19) United States
(12) Patent Application Publication
(10) Pub. No.: US 2010/0240967 A1
(43) Pub. Date: Sep. 23, 2010

(54) BELT WITH HEALTH MANAGEMENT FUNCTION

(76) Inventors: Seunghwan Kim, Daejeon-city (KR); Sun-Hee Park, Daejeon-City (KR); Soo-Young Oh, Daejeon-City (KR); Kwang-Ho Pyun, Seoul (KR); Kyung-Soo Kim, Seoul (KR); In-Sop Shim, Seoul (KR); Hyen-Oh La, Seoul (KR); Chang-Jin Choi, Seoul (KR); Se-Hong Kim, Seoul (KR)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/600,182

(22) PCT Filed: Mar. 27, 2008
(86) PCT No.: PCT/KR2008/001705

§ 371 Date: Nov. 13, 2009

(30) Foreign Application Priority Data
May 14, 2007 (KR) 10-2007-0046631

(51) Int. Cl. A61B 5/00

(52) U.S. Cl. 600/301

(57) ABSTRACT

A belt with a health management function includes a health information extraction module extracting health related information from various biomedical signals, a buckle housing the health information extraction module, and a strap connected to an end of the buckle. The health information extraction module includes a sensor module detecting various biomedical signals, and a process/extraction unit digitally processing the detected biomedical signals and extracting the health related information from the processed digital signals.

### TECHNICAL FIELD

The present invention relates to a belt, and more particularly, to a belt with a health management function.

This work was supported by the IT R&D program of MIC/IITA [2006-S-007-01, Ubiquitous Health Monitoring Module and System Development]

### BACKGROUND ART

Recently, a large attention to health is increasingly being paid, as per example, the ‘well-being’ trend. A method of monitoring health information in a simple and easy manner is being developed. The purpose of the present invention is to provide a simple and efficient health management by providing sensor information related to health through a belt that one wears everyday.

Among conventional methods related to the present invention, a first method discloses a pressure sensor of a spring type to measure an impedance precisely and a belt including the pressure sensor and which is attachable to the human body. Thus, body fat can be measured by measuring the impedance using the pressure sensor. The method can be used for the measurement of body fat based on the measured impedance and thus, providing a health analysis.

A second method discloses a belt, having a passometer and an LCD display attached to a buckle portion of the belt, to provide information such as calories consumed and walking speed based on the number of walked steps. However, the belt may be erroneous according to users. According to a third method, a scale mark is indicated on the rear side of a belt so that, when a user wears the belt, the user reads the scale mark to identify the waist circumference. Also, by inputting height and weight information, information regarding the physical condition of an individual, such as severely obese, obese, overweight, average weight, and underweight, can be provided according to the input values.

However, the above conventional methods produce large measurement errors because the measurements are manually performed. Also, the methods fail to provide a variety of biomedical information and the measured information is not properly managed.

### DISCLOSURE OF INVENTION

### Technical Problem

To solve the above and/or other problems, the present invention provides a belt with a health management function, wherein the belt can process detected biomedical information to extract health information and enable scientific and efficient management of the extracted health information.

### Technical Solution

According to an aspect of the present invention, a belt with a health management function comprises a health information extraction module extracting health related information from various biomedical signals, a buckle housing the health information extraction module, and a strap connected to an end of the buckle.

The health information extraction module comprises a sensor module detecting the various biomedical signals, and a process/extraction unit digitally processing the detected biomedical signals and extracting the health related information from the processed digital signals.

The health information extraction module further comprises a communication unit transmitting the health related information to the outside and a display unit displaying the health related information.

### Advantageous Effects

As described above, according to the present invention, a displacement sensor, a pressure sensor, an acceleration sensor, a position sensor, an electrocardiogram measurement sensor, an impedance measurement sensor, a blood pressure measurement sensor, and a temperature measurement sensor provide information regarding the health and position of a wearer by being included in a belt. Thus, health management can be conveniently and efficiently performed. As information regarding the position of a wearer is provided, the position of the wearer can be quickly identified in an emergency case.

Also, the electrocardiogram measurement module is included in the buckle so that an electrocardiogram using an external electrical potential sensor is possible. The impedance measurement module is included in the buckle so that a body fat rate and an abdominal fat rate of the human body can be obtained and thus, facilitating the management of abdominal obesity. Also, since the blood pressure measurement module is included in the buckle, blood pressure can be measured. Since the temperature measurement module is included in the buckle, the temperature of the human body can be measured. By including a wireless communication device and storing the information in a mobile phone or a PDA, not only temporary health information can be known, however, also health management and the prevention of obesity can be continuously performed.

Thus, by providing health information to a health-concerned individual, health management can be efficiently performed by providing information regarding to health. For an obese person, an efficient obesity management is possible by providing information regarding appropriate measurements to control the obesity. For a person having a cardiovascular disease, an unexpected cardiovascular problem can be quickly coped with by providing information regarding the heart rate and continuously monitoring the information. Furthermore, health can be continuously managed through a health management program including exercise.

### DESCRIPTION OF DRAWINGS

The above and other features and advantages of the present invention will become more apparent by describing in detail exemplary embodiments thereof with reference to the attached drawings in which:

FIG. 1 is a perspective view of a belt according to an embodiment of the present invention;

FIGS. 2A and 2B, respectively, are a front view and a rear view of the belt when a health information extraction module is closed;

FIGS. 3A and 3B, respectively, are a front view and a side view of the belt when the health information extraction module is opened;

FIG. 4 is a front view of when the health information extraction module is opened and in operation;

FIG. 5 illustrates examples of the health information displayed when selecting each menu tab in the health information extraction module of FIG. 4;

FIG. 6 illustrates an example of performing an electrocardiogram by connecting a lead wire of an electrical potential sensor to a connector of the health information extraction module;

FIG. 7 illustrates an example of the measurement of body fat when a lead wire of an electrical potential sensor is connected to a connector of the health information extraction module;

FIG. 8 illustrates an example of the measurement of temperature when a lead wire of an electrical potential sensor is connected to a connector of the health information extraction module;

FIG. 9 illustrates an example of the measurement of blood pressure when a lead wire of an electrical potential sensor is connected to a connector of the health information extraction module; and

FIG. 10 is a block diagram of the health information extraction module for the measurement and extraction of health information, according to an embodiment of the present invention.

### BEST MODE

According to an aspect of the present invention, a belt with a health management function comprises a health information extraction module extracting health related information from various biomedical signals, a buckle housing the health information extraction module, and a strap connected to an end of the buckle.

The health information extraction module comprises a sensor module detecting the various biomedical signals, and a process/extraction unit digitally processing the detected biomedical signals and extracting the health related information from the processed digital signals.

The health information extraction module further comprises a communication unit transmitting the health related information to the outside and a display unit displaying the health related information.

### Mode for Invention

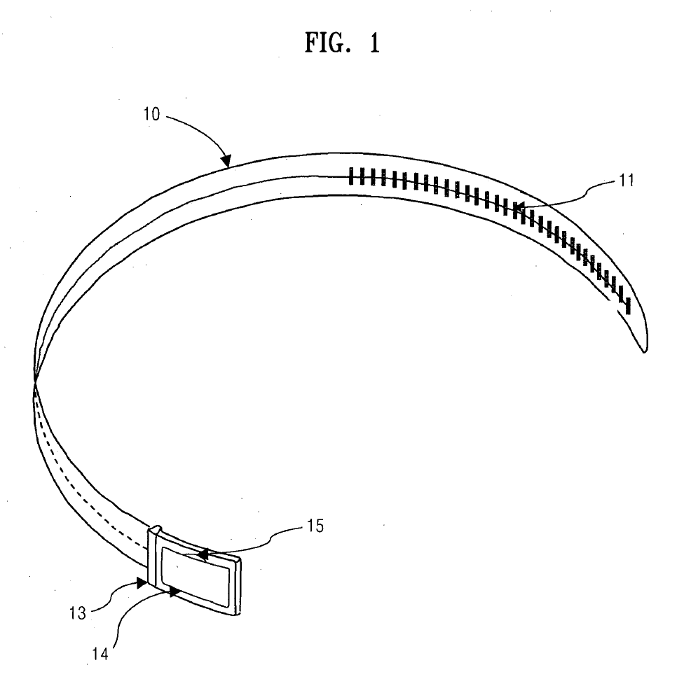

FIG. 1 is a perspective view of a belt according to an embodiment of the present invention. Referring to FIG. 1, the belt according to the present embodiment includes an electrical resistance body 11 provided on an inner surface of the belt and which can measure a waist circumference, a health information extraction module 14 extracting health information from a biomedical signal, a buckle 13 buckling and housing the health information extraction module 14, a groove 15 facilitating a rotary opening/closing of the health information extraction module 14 from the buckle 13, and a strap 10 supporting trousers.

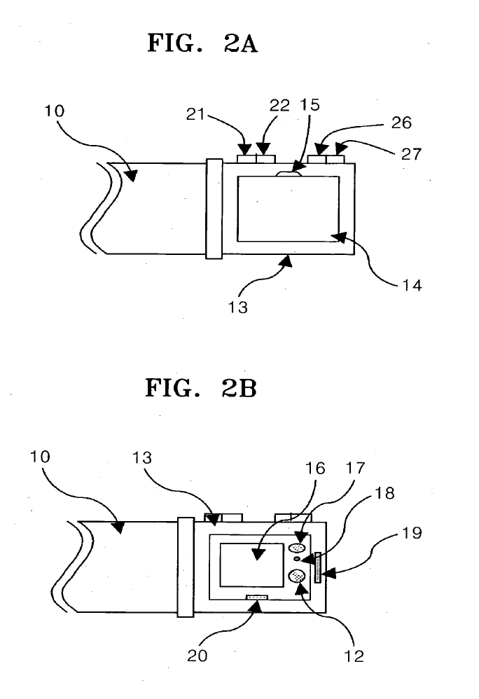

FIG. 2A is a front view of the belt when the health information extraction module 14 is closed. The buckle 13 fixes the strap 10 that supports trousers and simultaneously houses the health information extraction module 14. As shown in FIG. 2A, the groove 15 is formed in the buckle 13 to facilitate the rotation of the health information extraction module 14 from buckle 13. Also, the buckle 13 includes electrocardiogram measurement connector 21 and body fat measurement connector 22, which connect an electrical potential sensor and the health information extraction module 14, for measuring electrocardiogram and body fat. The buckle 13 includes blood pressure measurement connector 26 and temperature measurement connector 27, which connect a blood pressure sensor and a temperature sensor, for measuring blood pressure and temperature.

FIG. 2B is a rear view of the belt when the health information extraction module 14 is closed. Referring to FIG. 2B, a display unit 16 displaying a set screen of physical measurements and a result of measurements is provided in the health information extraction module 14. Also, a power button 17 for turning off the power when the belt is not in use to save power and a state LED 18 indicating the state of operation are provided in the health information extraction module 14 so that a user can see whether the belt is in use or not.

Since input of physical measurements, such as age, sex, and weight, is needed to determine body fat, an input key 12 is provided as a device to input the physical measurements. A pressure sensor 20 to measure an abdominal pressure is installed in a lower portion of the health information extraction module 14.

The pressure sensor 20 can be used to measure a waist circumference. The pressure sensor 20 enables a measurement of a waist circumference at a predetermined abdominal pressure and notifies a measurement position of the waist circumference as the state LED 18 flickers when a preset pressure is reached. A piezo-resistive silicon that outputs a change in electrical potential when receiving a force and other sorts of sensors can be used as the pressure sensor 20. A displacement sensor 19 detecting the resistance of the belt at the set abdominal pressure is provided so that a waist circumference is automatically measured by detecting the resistance of the resistance body 11.

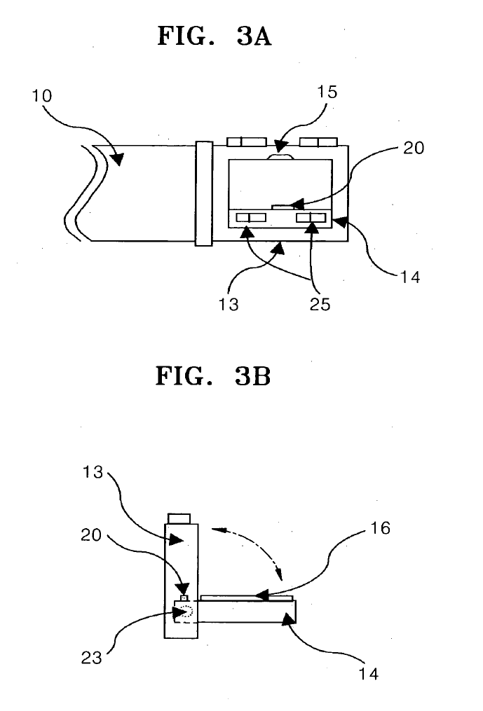

FIGS. 3A and 3B, respectively, are a front view and a side view of the belt when the health information extraction module 14 is opened. A rotary joint 23 connects the health information extraction module 14 and the buckle 13, so that the rotary opening and closing of the health information extraction module 14 from the buckle 13 is possible. The rotary joint 23 also transfers a measured value from the displacement sensor 19 to the health information extraction module 14. Also, a connection portion 25 connecting the connector 21, the connector 22, the connector 26, and the connector 27 to the health information extraction module 14, which respectively measure heart status, body fat, blood pressure, and temperature, is provided in the upper portion of the health information extraction module 14. Information can be checked through the display portion 16 and physical measurements can be input through the input key 12 when the health information extraction module 14 is opened.

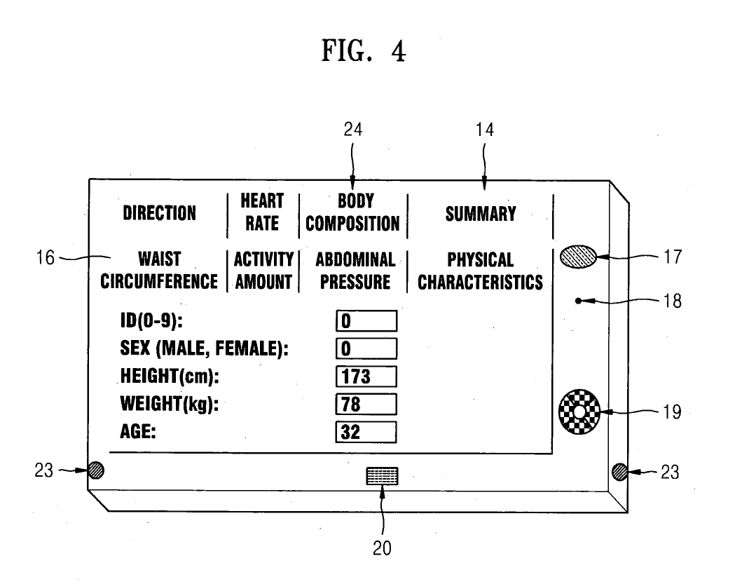

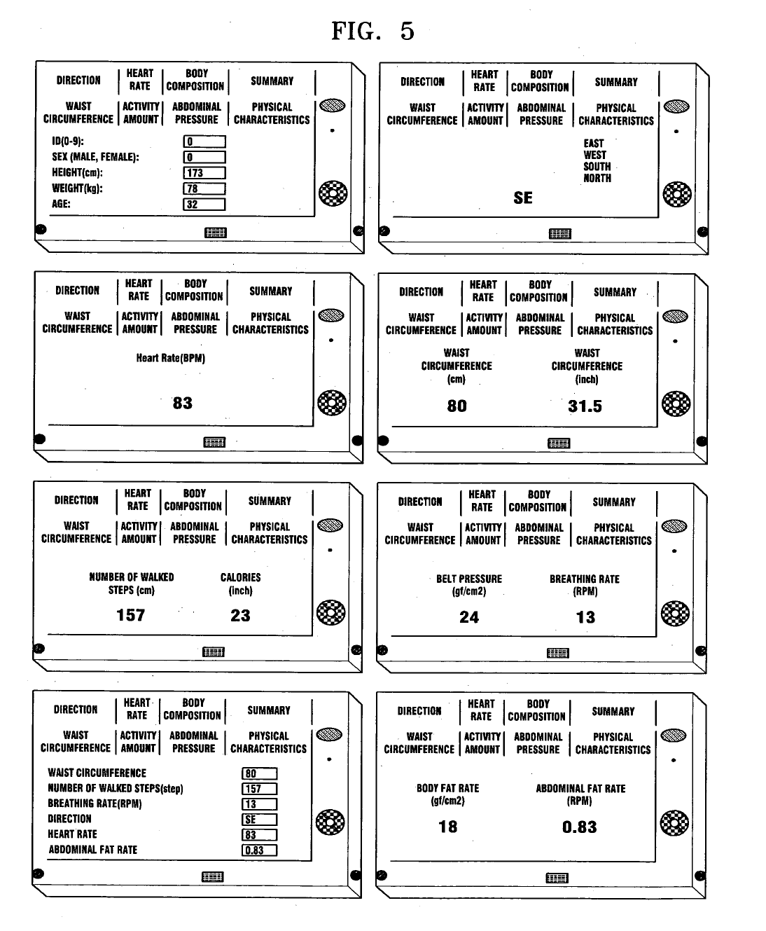

FIG. 4 is a front view of when the health information extraction module 14 is opened and in operation. Referring to FIG. 4, a menu tab 24 is provided in the upper portion of the display portion 16, in order to input physical measurements and check information about a waist circumference, an abdominal pressure, the amount of activity, a heart rate, a body composition, and a position. Thus, the measured health information can be checked by selecting each menu tab. FIG. 5 shows health information when each menu tab is selected in the health information extraction module of FIG. 4. In FIG. 5, in a clockwise direction from the top left corner, a case of selecting a physical characteristics tab, a case of selecting a direction tab, a case of selecting a waist circumference tab, a case of selecting an abdominal pressure tab, a case of selecting a body composition tab, a case of selecting a summary tab, a case of selecting an activity measurements tab, and a case of selecting a heart rate tab, are shown.

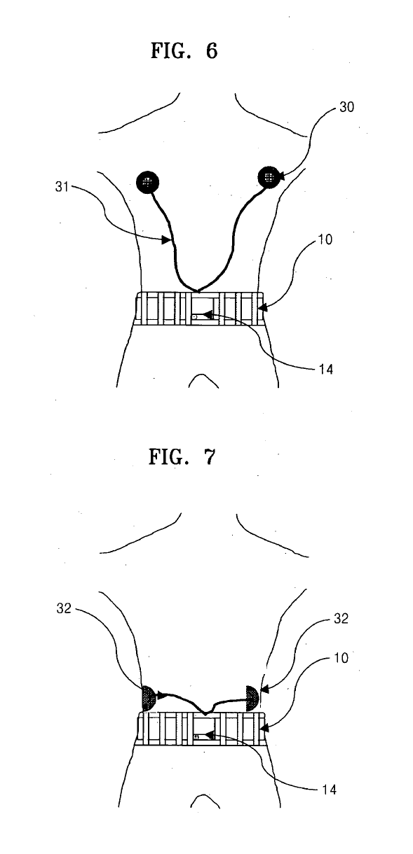

FIG. 6 illustrates an example of performing an electrocardiogram by connecting a lead wire 31 of an electrical potential sensor 30 to the electrocardiogram measurement connector 21 of the health information extraction module 14. Referring to FIG. 6, an electrical activity of the heart is detected by the electrical potential sensor 30 and transferred to the health information extraction module 14 through the lead wire 31 so that the health information extraction module 14 performs the electrocardiogram to obtain a heart rate.

FIG. 7 illustrates an example of the measurement of body fat when a lead wire of an electrical potential sensor 32 is connected to the body fat measurement connector 22 of the health information extraction module 14. Referring to FIG. 7, a body fat rate is measured by applying a weak current to the skin surface to measure an impedance.

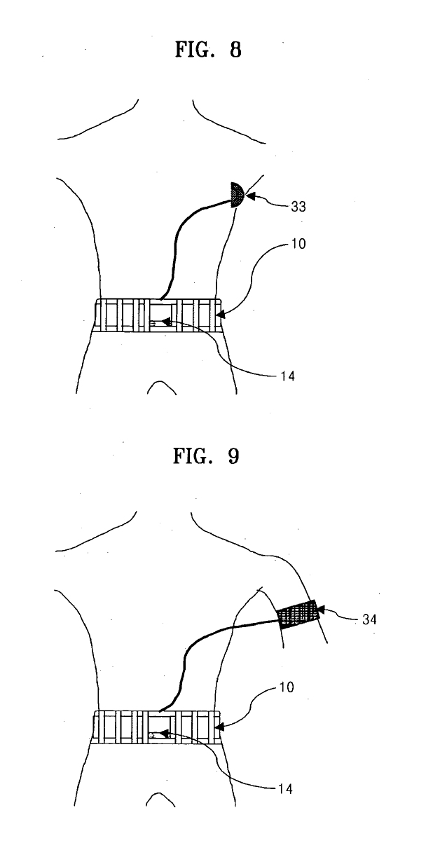

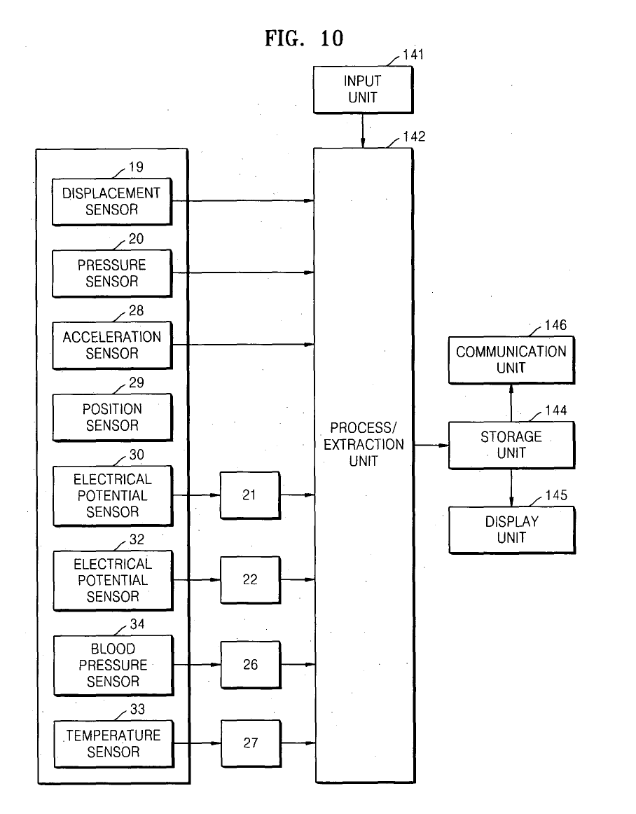

FIG. 8 illustrates an example of the measurement of temperature when a lead wire of an electrical potential sensor 33 is connected to the temperature measurement connector 27. FIG. 9 illustrates an example of the measurement of blood pressure when a lead wire of an electrical potential sensor 34 is connected to the blood pressure measurement connector 26 of the health information extraction module 14. FIG. 10 is a block diagram of the health information extraction module for the measurement and extraction of health information, according to an embodiment of the present invention.

The health information extraction module 14, as shown in FIG. 10, includes a sensor module 141 detecting various biomedical signals, a process/extraction unit 142 processing the biomedical signals detected by the sensor module 141 and extracting health information from the processed biomedical signals, an input unit 143 through which physical measurements of a user are input, a storage unit 144 storing the extracted health information, a display unit 145 displaying the health information or allowing input of personal physical measurements, and a communication unit 146 transmitting the health information to the outside, for example, a PC, a PDA, or a mobile phone.

The sensor module 141 is a module formed of the displacement sensor 19, an acceleration sensor 28, the pressure sensor 20, a position sensor 29, the electrical potential sensors 30 and 32, the blood pressure sensor 34, and the temperature sensor 33. The acceleration sensor 28 is for extracting the amount of activity of a user using information regarding the number of walked steps and consumed calories. The position sensor 29 is for extracting information regarding the present position of a user.

The process/extraction unit 142 performs a predetermined signal process including filtering with respect to a biomedical signal measured from the sensors and extracts health information through a health information extraction algorithm by digitizing the processed signal. The extracted health information is stored in the storage unit 144 and provided to a user through a display device such as an LCD by the display unit 145. Also, the communication unit 146 can transmit information to a PDA, a mobile phone, or a PC of a wearer using wired/wireless communication devices such as a wired communication using a USB or a wireless communication using Bluetooth.

While this invention has been particularly shown and described with reference to preferred embodiments thereof, it will be understood by one skilled in the art that various changes in form and details may be made therein without departing from the spirit and scope of the invention as defined by the appended claims.

As described above, according to the present invention, a displacement sensor, a pressure sensor, an acceleration sensor, a position sensor, an electrocardiogram measurement sensor, an impedance measurement sensor, a blood pressure measurement sensor, and a temperature measurement sensor provide information regarding the health and position of a wearer by being included in a belt. Thus, health management can be conveniently and efficiently performed. As information regarding the position of a wearer is provided, the position of the wearer can be quickly identified in an emergency case.

Also, the electrocardiogram measurement module is included in the buckle so that an electrocardiogram using an external electrical potential sensor is possible. The impedance measurement module is included in the buckle so that a body fat rate and an abdominal fat rate of the human body can be obtained and thus, facilitating the management of abdominal obesity. Also, since the blood pressure measurement module is included in the buckle, blood pressure can be measured. Since the temperature measurement module is included in the buckle, the temperature of the human body can be measured. By including a wireless communication device and storing the information in a mobile phone or a PDA, not only temporary health information can be known, however, also health management and the prevention of obesity can be continuously performed.

Thus, by providing health information to a health-concerned individual, health management can be efficiently performed by providing information regarding to health. For an obese person, an efficient obesity management is possible by providing information regarding appropriate measurements to control the obesity. For a person having a cardiovascular disease, an unexpected cardiovascular problem can be quickly coped with by providing information regarding the heart rate and continuously monitoring the information. Furthermore, health can be continuously managed through a health management program including exercise.

1. A belt with a health management function, the belt comprising:
a health information extraction module extracting health related information from various biomedical signals;
a buckle housing the health information extraction module; and
a strap connected to an end of the buckle.

2. The belt of claim 1, wherein the health information extraction module comprises:
a sensor module detecting the various biomedical signals; and
a process/extraction unit digitally processing the detected biomedical signals and extracting the health related information from the processed digital signals.

3. The belt of claim 2, wherein the health information extraction module further comprises:
a communication unit transmitting the health related information to the outside; and
a display unit displaying the health related information.

4. The belt of claim 2, wherein the sensor module includes a pressure sensor so that the process/extraction unit extracts abdominal pressure information.

5. The belt of claim 4, wherein the process/extraction unit further extracts waist circumference information from the extracted abdominal pressure information.

6. The belt of claim 2, wherein resistors are provided on an inner surface of the strap, and a displacement sensor is included in the sensor module to detect a change in the resistance of the resistors so that the process/extraction unit extracts waist circumference information.

7. The belt of claim 2, wherein an acceleration sensor is provided in the sensor module so that the process/extraction unit extracts information regarding the number of walked steps and information regarding consumed calories.

8. The belt of claim 2, wherein a position sensor is provided in the sensor module so that the process/extraction unit extracts information regarding the position of a user.

9. The belt of claim 2, wherein an electrical potential sensor for an electrocardiogram is provided in the sensor module and the electrical potential sensor is connected to a connector, for the electrocardiogram, provided in the health information extraction module, through a lead wire, so that the process/extraction unit extracts information regarding the electrocardiogram of a user.

10. The belt of claim 2, wherein an electrical potential sensor for a measurement of body fat is provided in the sensor module and the electrical potential sensor is connected to a connector, for the measurement of body fat, provided in the health information extraction module, through a lead wire, so that the process/extraction unit extracts information regarding body fat of a user.

11. The belt of claim 2, wherein a temperature sensor for a measurement of temperature is provided in the sensor module and the temperature sensor is connected to a connector, for the measurement of temperature, provided in the health information extraction module, through a lead wire, so that the process/extraction unit extracts information regarding the temperature of a user.

12. The belt of claim 2, wherein a blood pressure sensor for a measurement blood pressure is provided in the sensor module and the electrical potential sensor is connected to a connector, for the measurement of blood pressure, provided in the health information extraction module, through a lead wire, so that the process/extraction unit extracts information regarding the blood pressure of a user.

13. The belt of claim 1, wherein a groove is formed in a surface of the buckle to facilitate a rotary opening/closing of the health information extraction module from the buckle.
